(19) United States
(12) Patent Application Publication
(10) Pub. No.: US 2013/0305600 A1
(43) Pub. Date: Nov. 21, 2013

(54) HANDHELD VIBRATING POLLINATORS AND POLLINATION METHODS

(71) Applicant: VEGIBEE, LLC, St. Louis, MO (US)

(72) Inventor: William Michael Whaley, St. Louis, MO (US)

(73) Assignee: VEGIBEE, LLC, St. Louis, MO (US)

(21) Appl. No.: 13/768,744

(22) Filed: Feb. 15, 2013

Related U.S. Application Data
(60) Provisional application No. 61/648,231, filed on May 17, 2012.

(51) Int. Cl. A01H 1/02

(52) U.S. Cl. CPC A01H 1/025
USPC 47/1.41

(57) ABSTRACT

Exemplary embodiments are disclosed of handheld vibrating pollinators. Also disclosed are exemplary embodiments of pollination methods. In an exemplary embodiment, a pollinator generally includes a pollen capture device (e.g., a spoon, etc.) for capturing pollen released from a flower. The pollinator also includes a handle portion operable for generating vibrations and at least one member (e.g., one or more wands, etc.) configured to be installed on the handle portion. The at least one member is operable for transferring the vibrations generated by the handle portion to a flower for causing the flower to release pollen for capture by the pollen capture device.

### CROSS-REFERENCE TO RELATED APPLICATION

This patent application is a U.S. non-provisional patent application which claims priority to and benefit of U.S. provisional patent application No. 61/648,231 filed May 17, 2012. The disclosure of the application identified in this paragraph is incorporated herein by reference in its entirety.

### FIELD

The present disclosure relates to handheld sonic vibrating pollinators and pollination methods.

### BACKGROUND

This section provides background information related to the present disclosure which is not necessarily prior art.

Many people have home gardens in their backyards, etc. Common vegetable plants found in such home gardens include tomatoes, cucumbers, zucchini, squash, pumpkin, watermelon, peppers, beans, peas, eggplant, etc.

### SUMMARY

This section provides a general summary of the disclosure, and is not a comprehensive disclosure of its full scope or all of its features.

Exemplary embodiments are disclosed of handheld vibrating pollinators. Also disclosed are exemplary embodiments of pollination methods. In an exemplary embodiment, a pollinator generally includes a pollen capture device (e.g., a spoon, etc.) for capturing pollen released from a flower. The pollinator also includes a handle portion operable for generating vibrations and at least one member (e.g., one or more wands, etc.) configured to be installed on the handle portion. The at least one member is operable for transferring the vibrations generated by the handle portion to a flower for causing the flower to release pollen for capture by the pollen capture device.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a perspective view of an exemplary embodiment of a pollinator (broadly, a gardening appliance or device) and showing various components thereof including a handle portion, interchangeable wands (broadly, interchangeable vibrational transfer members), spoon (broadly, pollen capture device), and charging base unit;

FIG. 2 is an exploded perspective view of the pollinator shown in FIG. 1;

FIGS. 3A and 3B are perspective views showing the exemplary manner by which one wand may be removed from and the other wand installed onto the handle portion according to an exemplary embodiment;

FIGS. 4 and 5 illustrate two different examples of wands having different lengths (110 millimeters (mm) and 180 mm, respectively) that may be used with the pollinator shown in FIG. 1 according to exemplary embodiments, where the dimensions (shown in millimeters) are provided for purposes of illustration only;

FIG. 6 illustrates an example of a spoon that may be used to collect or capture excess released pollen released from a flower while using the pollinator shown in FIG. 1 according to an exemplary embodiment, where the dimensions (in millimeters) are provided for purposes of illustration only;

FIGS. 7 and 8 show the pollinator shown in FIG. 1 being used with the short wand and the spoon according to a first exemplary embodiment of a pollination method;

FIG. 9 shows the pollinator shown in FIG. 1 being used with the long wand without the spoon according to a second exemplary embodiment of a pollination method;

FIG. 10 is a perspective view of another exemplary embodiment of a pollinator (broadly, a gardening appliance or device) and showing various components thereof including a handle portion, wand (broadly, interchangeable vibrational transfer member), spoon (broadly, pollen capture device), and base unit;

FIG. 11 shows the various components of the pollinator shown in FIG. 10, and also illustrating an AA battery that provides electrical power for operating the pollinator in this exemplary embodiment; and

FIG. 12 illustrates examples of a wand, spoon, handle portion, and base unit that may be used with the pollinator shown in FIG. 10 according to exemplary embodiments, where the dimensions (shown in millimeters) are provided for purposes of illustration only.

### DETAILED DESCRIPTION

Example embodiments will now be described more fully with reference to the accompanying drawings.

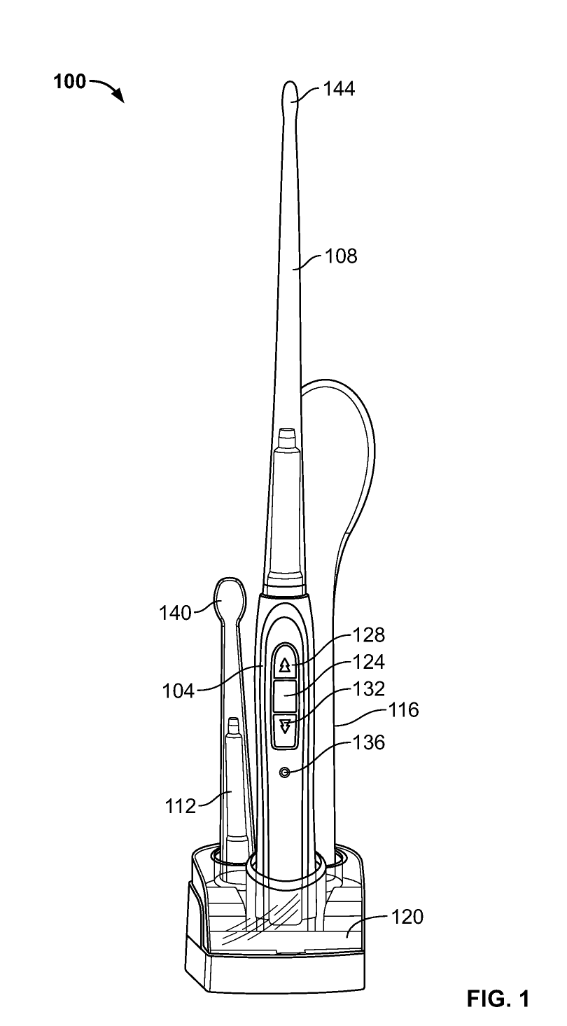

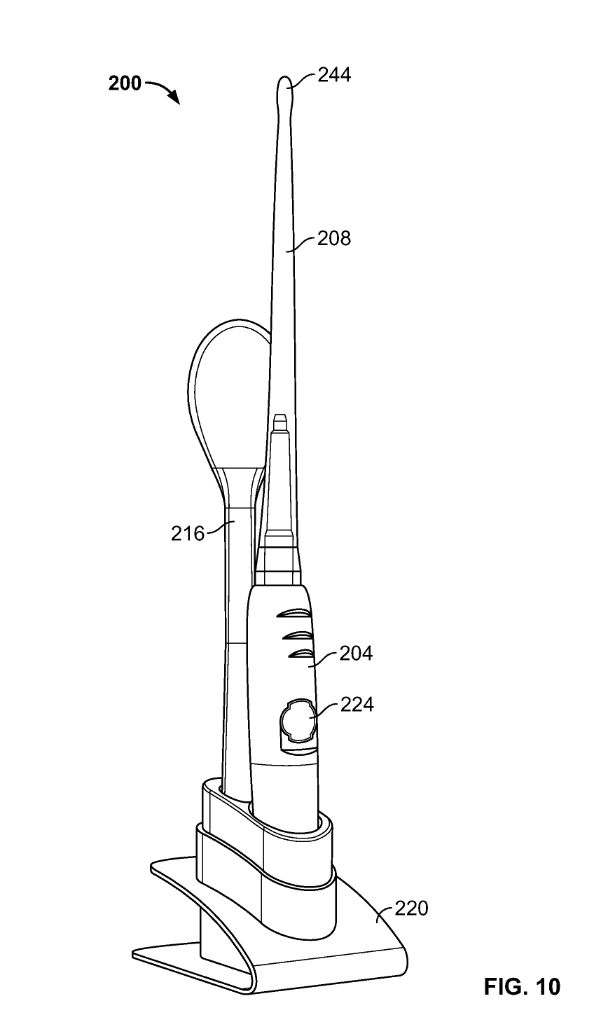

The inventor hereof discloses exemplary embodiments of gardening appliances, devices, systems, and methods that allow improved crop yields in home gardens, green houses, hydroponic gardens, etc. In exemplary embodiments, a handheld electric vibrating device or pollinator (e.g., pollinator 100 shown in FIG. 1, pollinator 200 shown in FIG. 10, etc.) is operable for generating sonic vibrations (e.g., more than 30,000 vibrations per minute, 29,000 to 44,000 vibrations per minute, 38,000 vibrations per minute, etc.). The sonic vibrations generated by the pollinator imitate high frequency vibrations made by a bee's wings during pollination. The inventor hereof has found that the use of sonic vibrations is very effective in causing the release of pollen from the anthers of a flower's stamen (or male part) to pollinate the flower's stigma (or female part).

As disclosed herein, the inventor's pollinators are operable for creating sonic vibrations that cause a gentle release of pollen from the flower onto a specially designed collection device, e.g., spoon, etc. In a matter of seconds, enough pollen may be collected or captured by the spoon to pollinate four or five flowers by dipping the flower stigma from the same plant into the collected pollen. A home gardener, greenhouse grower, hydroponic grower, flower (e.g., roses, orchids, etc.) grower, etc. may then use the captured excess pollen to pollinate additional flowers on the same plant that released the captured pollen or to pollinate flowers on a different plant of the same type. By using the inventor's pollinators and methods disclosed herein, average garden yield might be significantly increased, e.g., by more than 30% annually, etc.

By way of example, the inventor's pollinators and methods may be used to assist pollination on a wide variety of vegetable plants, such as tomatoes (e.g., cherry tomatoes, globe tomatoes, beefsteak tomatoes, etc.), peppers, beans, peas, eggplant, etc. Tomatoes, peppers, beans, peas, and eggplant are self-pollinating plants that possess the “perfect flower” with both male and female reproductive parts in the same flower. The inventors' pollinators may be used to help overcome low crop yields in these plants caused by shrinking bee populations and home gardens sheltered from the wind. The inventors' pollinators may also be used for pollinating flowers, such as roses, orchids, etc.

Commercial growers commonly use costly large vibrating devices to pollinate their vegetable producing plants. But the inventor hereof has recognized that commercial growers allow excess pollen to wastefully fall to the ground. Thus, the inventor hereof has developed and discloses exemplary embodiments of pollinators or pollination systems and methods in which all or a substantial amount (e.g., a majority, most, 50% to 100%, etc.) of the released excess pollen is collectible or captured in a specially designed pollen capture device, e.g., spoon, etc. In addition, the inventor's pollinators may include an internal power source (e.g., rechargeable or non-rechargeable battery(ies), etc.), such that there are not any electrical cords that have to be managed (e.g., avoid tripping over, etc.) while using the pollinator in a garden. In exemplary embodiments, the handle portion includes a battery compartment for one or more rechargeable or non-rechargeable batteries for electrically powering the pollinator.

In exemplary embodiments disclosed herein, a handheld vibrating device or pollinator may include two differently configured vibrating wands that are interchangeably mountable or installable onto the handle portion of the pollinator. The different wands are configured differently (e.g., different lengths, differently sized tips or end portions, etc.). In other exemplary embodiments disclosed herein, only one wand or more than two wands may be provided along with a pollinator. Each wand may preferably be configured (e.g., made of relatively stiff material, etc.) to dampen or reduce the amplitude of the sonic vibrations traveling therethrough. If the amplitude of the sonic vibrations is too high, the flower may be damaged when the vibrating wand is positioned against the flower's stamen to cause the release of pollen.

In those exemplary embodiments having two or more wands, the differently configured wands or members may be used in a respective one of two different pollinating methods. In a first pollination method disclosed herein, the shorter wand is installed to the handle portion, and a spoon is used to capture or collect the excess released pollen that falls into and on the spoon. The captured pollen may then be transferred and used to pollinate additional flowers. In a second pollination method, the longer wand is installed to the handle portion and used to cause pollination of small delicate flowers, for example, by touching the vibrating end portion of the wand to the stigmas of flowers of cherry tomatoes, etc. But the spoon is not used to capture or transfer the released pollen in this second method. The longer wand may also be used for plants that are high up or very low where the extra length of the wand makes it easier on the user. Advantageously, the inventor's exemplary embodiments of systems and methods for pollinating a garden may only take about 10 minutes a day and 3 times a week to yield significant increases in garden yield, e.g., increased garden yield by at least 30% annually.

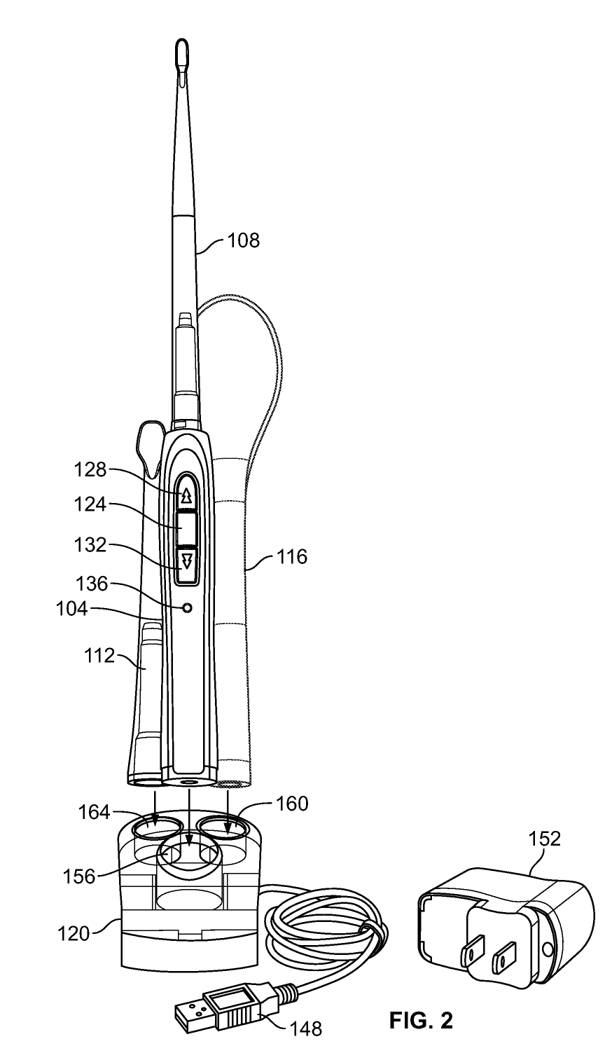

With reference to the figures, FIGS. 1 and 2 illustrate an exemplary embodiment of a handheld sonic vibrating device or pollinator 100 (broadly, a gardening appliance or device) embodying one or more aspects of the present disclosure. As shown in FIG. 1, the pollinator 100 includes various components including a vibration generator or handle portion 104 for generating sonic vibrations. The pollinator 100 also includes two interchangeable wands or members 108, 112 for transferring the sonic vibrations to a flower to cause pollen to be released. The pollinator 100 also includes a spoon 116 (broadly, pollen capture or collection device) and a base unit 120.

The handle portion 104 includes an internal mechanism or means for causing the sonic vibrations. By way of example, the handle portion 104 may be substantially similar to an electric toothbrush handle. In operation, the handle portion 104 is operable for generating sonic vibrations, e.g., 29,000 to 44,000 vibrations per minute. The handle portion 104 may include vibration dampening or reduction means. The handle portion 104 may be made of ROHS certified material (e.g., plastic, etc.).

The handle portion 104 includes an on/off button 124, buttons 128, 132 with respective up and down arrows for changing the vibration speed. There is an LED light indicator 136 for indicating when the device 100 is on or being charged. The handle portion 104 may include other means for selecting from amongst various vibration speeds, e.g., five vibration speed settings from 29,000 to 44,000 vibrations per minute. The selection of a vibration speed may be based on the type of flower, how fragile and delicate the particular type of flower is, the pollination method used, etc.

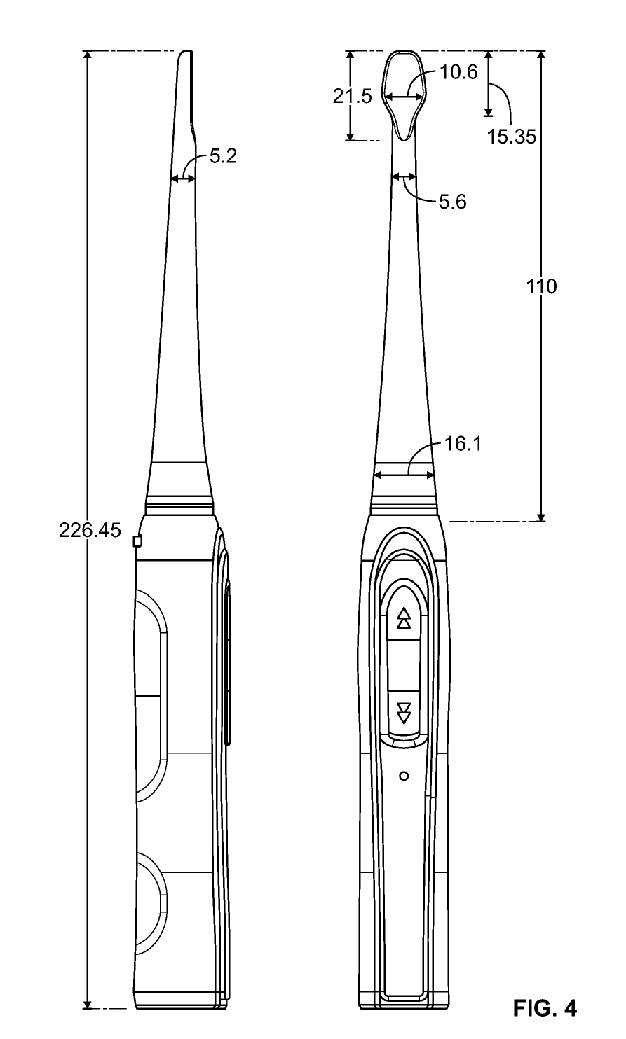

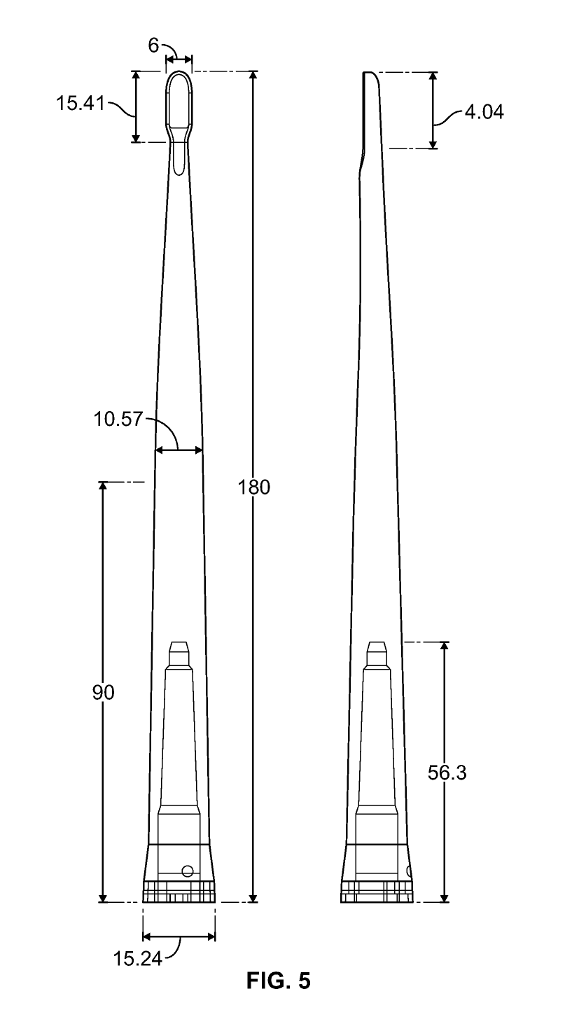

The wands 108, 112 are interchangeably mountable or attachable to the handle portion 104. By way of example, the shorter wand 112 may be 11 centimeters long, while the longer wand may be 18 centimeters long. FIGS. 4 and 5 illustrate two different examples of wands having different lengths (110 millimeters and 180 millimeters, respectively) that may be used with the pollinator 100 according to exemplary embodiments. The dimensions (in millimeters) are provided in FIGS. 4 and 5 solely for the purpose of illustration.

In addition to having different lengths, the wands 108, 112 may also have differently configured tips or end portions, e.g., different shapes and/or sizes, etc. For example, the shorter wand 112 may have a tip or end portion 140 that is larger (e.g., wider, longer, etc.) than the tip or end portion 144 of the longer wand 108. The wand tips 140, 144 may have textured, roughened, or matt surfaces. This, in turn, may help increase friction between the wand end portions 140, 144 and the flower against which the end portions 140, 144 have been positioned and help the wands 108, 112 remain in contact with the flower. The wands 108, 112 may also be configured (e.g., made of relatively stiff material, etc.) to dampen or reduce the amplitude of the sonic vibrations traveling along or through the vibrating wands 108, 112. This amplitude reduction helps prevent or inhibit damage to the flower from when the vibrating wands 108, 112 are positioned against the flower for causing the release of pollen. The selection of which wand to use may be based on the type of flower and pollination method used.

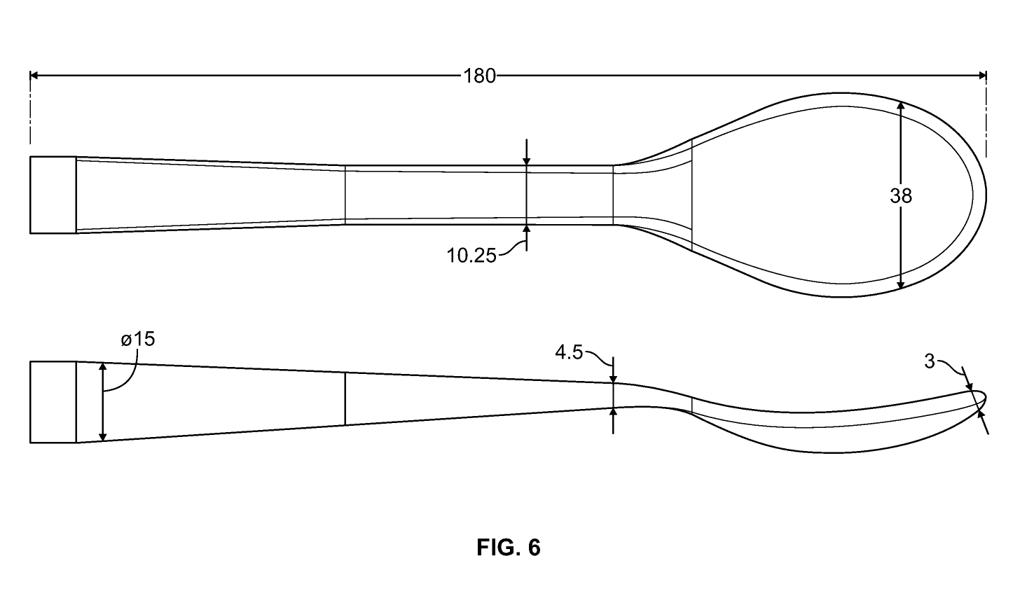

The spoon 116 is used to collect or capture excess released pollen released from a flower while using the pollinator 100. By way of example only, the spoon 116 may be 18 centimeters long and made of FDA food grade plastics, etc. The spoon 116 may be darker in color (e.g., black glossy color, etc.) than the pollen, which is relatively light in color. By having the spoon 116 darker in color than pollen, a user is more easily able to determine whether and when the spoon 116 has captured pollen. The spoon 116 may also be made of a material that allows natural sticky pollen to adhere and stick to the spoon 116. FIG. 6 illustrates an example of the spoon 116 that may be used to collect or capture excess released pollen released from a flower while using the pollinator 100. The dimensions (in millimeters) are provided in FIG. 6 solely for the purpose of illustration. Alternative embodiments may include a differently configured pollen capture device.

The handle portion 104 of the pollinator 100 may include a rechargeable power source (e.g., nickel metal hydride (Ni-MH) battery, etc.) that is rechargeable via the electric recharging base unit 120. The base unit 120 is configured to be electrically powered by way of a USB connector 148 (FIG. 2), which is pluggable into a USB port of a computer or an electric wall AC adapter 152 (FIG. 2) having a UL certified plug. In this example embodiment, the pollinator 100 may be configured such that the charging mode is via electromagnetic induction and the power supply is AC 100-240 Volts, 50 Hertz. The initial charging time for the pollinator 100 may be 12 hours when the USB connector 148 is used with the AC adapter 152, or 16 hours when the USB connector 148 is used with a computer. The handle portion 104 includes an LED 136 (e.g., green LED, etc.) or other light source to indicate that the device 100 is charging (e.g., when light is blinking, etc.) or that the device 100 is on (e.g., when LED is illuminated but not blinking, etc.).

The base unit 120 also includes three openings 156, 160, 164 as shown in FIG. 2. The openings 156, 160, 164 are configured for respectively receiving lower portions of the handle portion 104, spoon 116, and whichever wand 108, 112 is installed on the handle portion 104. FIG. 1 illustrates the exemplary manner in which the handle portion 104, spoon 116, and wand 112 may be held upright with their lower portions within the respective openings 156, 160, 164 when the pollinator 100 is not in use. Accordingly, the base unit 120 thus provides a compact and easy way of storing the components of the pollinator 100.

Continuing with this example, the pollinator 100 shown in FIG. 1 may have an overall length of about 23 centimeters when the shorter 11 centimeter wand 112 is installed on the handle portion 104. The pollinator 100 may have an overall length of about 30 centimeters (e.g., 298 millimeters, etc.,) when the longer 18 centimeter wand 108 is installed on the handle portion 104. The handle portion 104 weight may be about 61 grams, while the charger or base unit 120 weight is about 81 grams. The pollinator 100 may be provided as a packaged product with product packaging and operating instructions. The specific dimensions, numerical values, and specific materials disclosed for exemplary embodiment herein are example in nature and do not limit the scope of the present disclosure, as other exemplary embodiments may be configured differently, such as larger, smaller, and/or made from different materials.

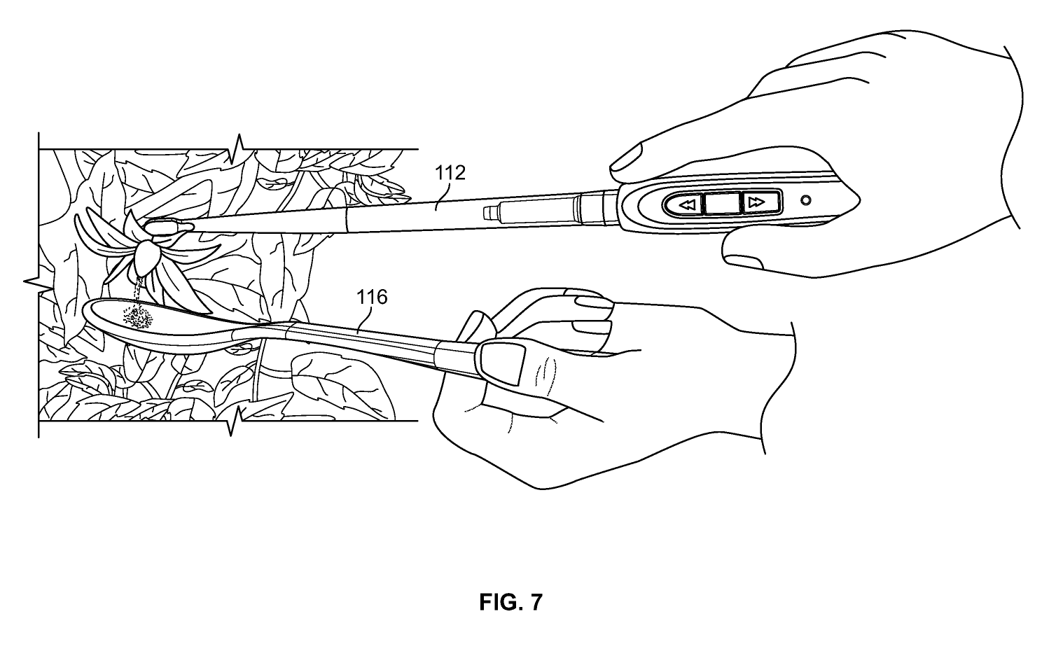

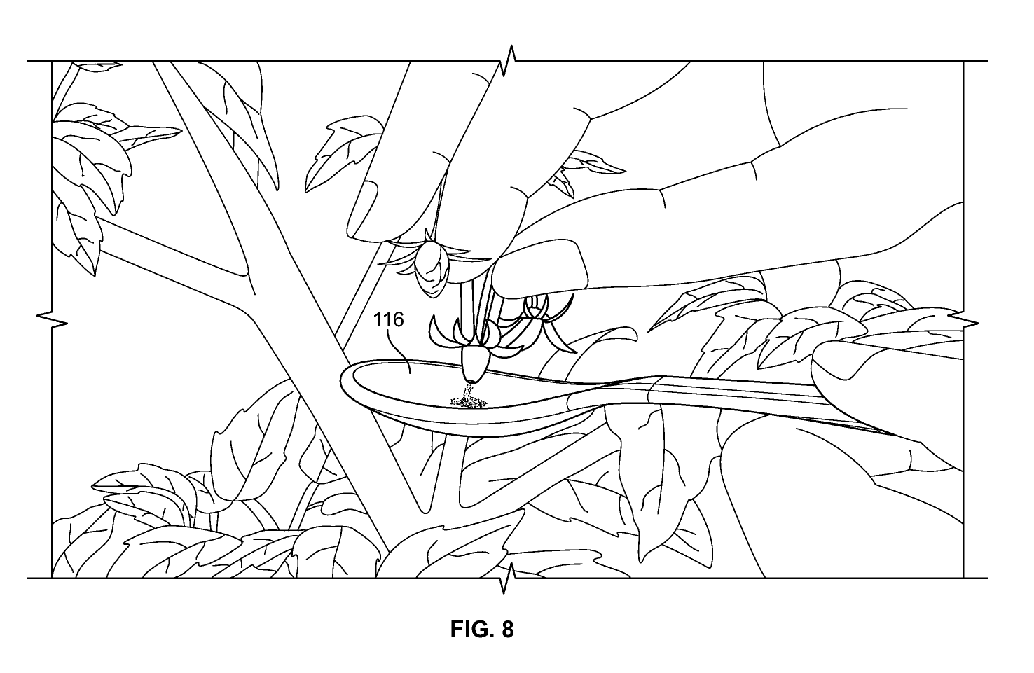

With reference to FIGS. 7 and 8, the pollinator 100 is shown being used with the shorter wand 112 and the spoon 116 according to a first exemplary embodiment of a pollination method. This example method generally includes placing the vibrating wand 112 on the stem above a flower cluster and tilting the flowers downward with the wand 112. The spoon 116 is placed under flower cluster (FIG. 7). The pollinator 100 is then turned on high vibration for 4 seconds, such that the flowers at peak freshness will release pollen. The pollinator 100 is then turned off.

As shown in FIG. 8, the stigma from multiple flowers may then be gently dipped in the pollen collected or captured on the spoon 116. The steps described above in regard to FIG. 7 may then be repeated to capture and collect more pollen. If the flower is pollinated, the flower will close the next day. The spoon 116 may be rinsed to remove remaining pollen before using the pollinator 100 for pollinating a different plant species. After use, the pollinator 100 may be turned off and returned to the charging base 120.

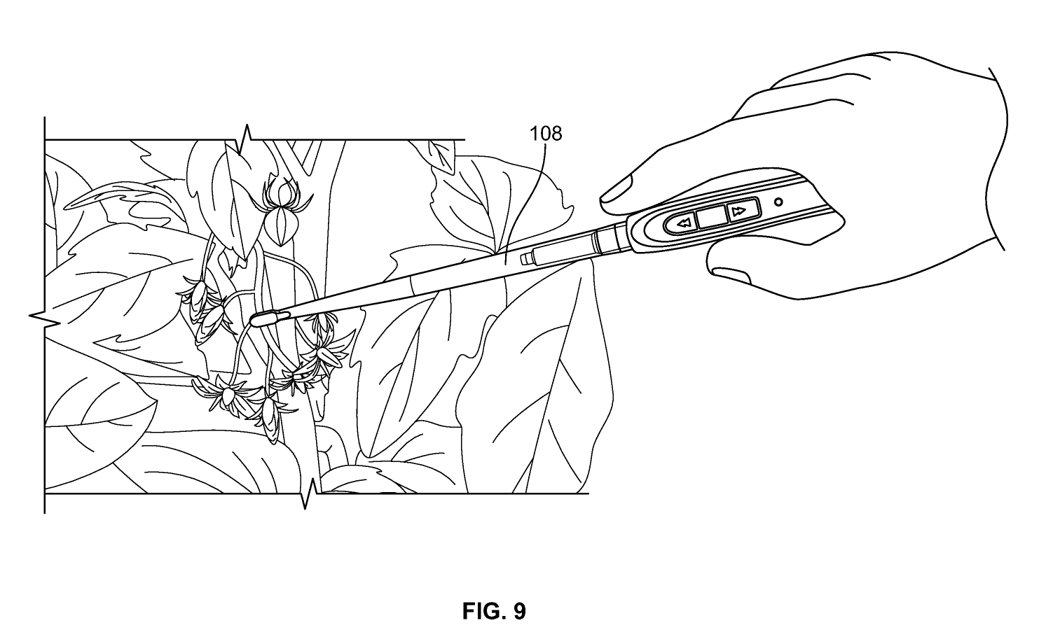

FIG. 9 shows the pollinator 100 being used with the longer wand 108 without the spoon 116 according to a second exemplary embodiment of a pollination method, which may be used on plants with small flowers like cherry tomatoes. This second example method generally includes placing the wand 108 on the stem above the flower cluster and tilting the flowers downward with the wand 108 as shown in FIG. 9. The pollinator 100 may then be turned on slow vibration for 4 seconds and for repeated 4-second uses as necessary. The pollinator 100 is then turned off and returned to the charging base 120.

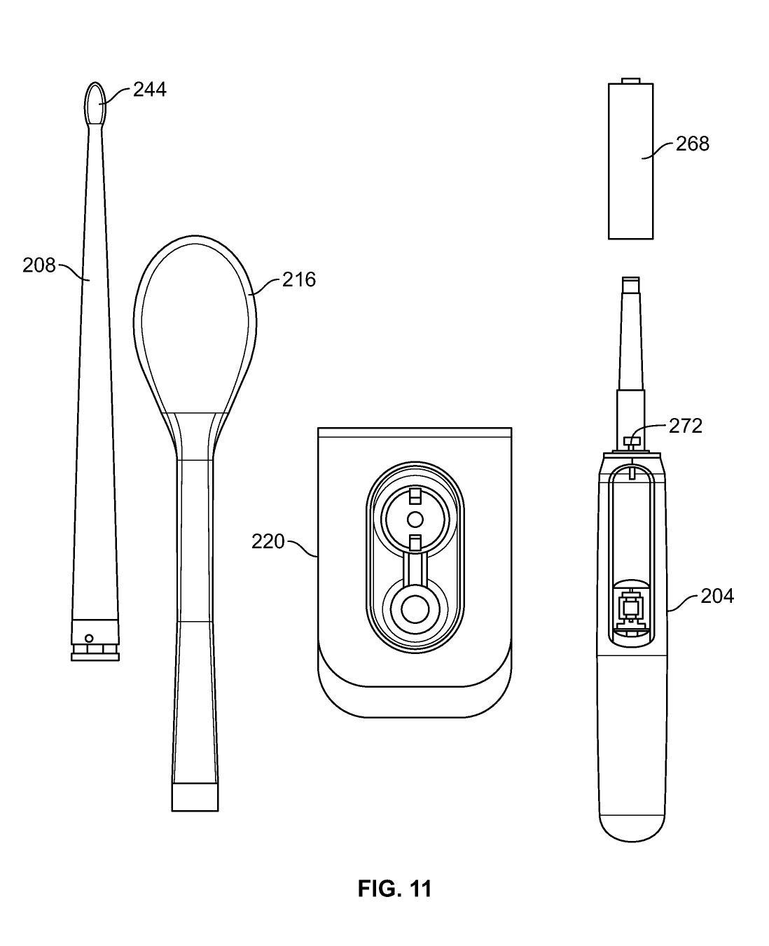

FIGS. 10 and 11 illustrate another exemplary embodiment of a handheld sonic vibrating device or pollinator 200 (broadly, a gardening appliance or device) embodying one or more aspects of the present disclosure. As shown in FIG. 10, the pollinator 200 includes various components including a vibration generator or handle portion 204 for generating sonic vibrations. The pollinator 200 also includes a spoon 216 (broadly, pollen capture or collection device) and a base unit 220.

In this example, the pollinator 200 includes only one wand 208, which may be fixedly or removably attached to the handle portion 204. In operation, the wand 208 is operable for transferring the sonic vibrations to a flower to cause pollen to be released. The wand 208 may have a tip or end portion 244 having textured, roughened, or matt surfaces, which may help the wand 208 remain in contact with the flower of the plant after being positioned against the flower. The wand 208 may be configured (e.g., made of relatively stiff material, etc.) to dampen or reduce the amplitude of the sonic vibrations traveling along or through the vibrating wand 208. This amplitude reduction helps prevent or inhibit damage to the flower when the vibrating wand 208 is positioned against the flower to cause the release of pollen.

In this example, the pollinator 200 is battery powered via a single AA battery 268. Accordingly, the base unit 220 in this example is not a charging unit. Alternative embodiments may include other means (e.g., rechargeable batteries, etc.) for providing electrical power for operating the pollinator 200.

Also in this example, the pollinator 200 is a single speed pollinator. Accordingly, the handle portion 204 includes an on/off button 224 but does not include buttons up and down arrows for changing the vibration speed.

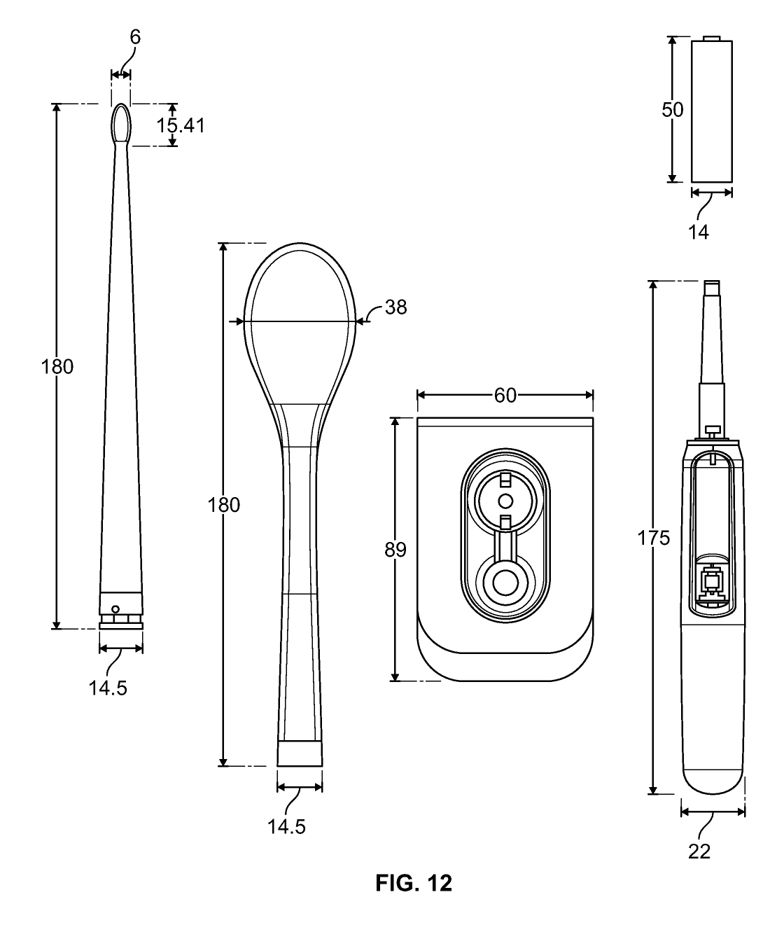

By way of example only, the single speed of the sonic vibrations produced by the pollinator 200 may be 38,000 vibrations per minute. The vibrating wand 208 may have a length of about 18 centimeters and/or other dimensions similar to those shown in FIG. 5 for the wand 108. The handle portion 204 may have a maximum width of 21.85 millimeters. In addition, the overall length of the handle portion 204 with the wand 208 attached thereto may be 30.1 centimeters. The spoon 216 may have similar dimensions to those shown in FIG. 6 for the spoon 116. FIG. 12 illustrates examples of a wand, spoon, handle portion, and base unit that may be used with the pollinator 200 shown in FIG. 10 according to exemplary embodiments. The specific dimensions and numerical values disclosed in this paragraph are example in nature and do not limit the scope of the present disclosure, as other exemplary embodiments may be configured differently, such as larger, smaller, multispeed, produce faster or slower vibrations, and/or made from different materials.

A description will now be provided of exemplary operating instructions and steps that may be taken while using an exemplary embodiment of a pollinator disclosed herein. The pollinator may be turned on by pressing the on switch located on the handle portion to thereby start vibrating the wand. If the pollinator has multiple speeds (e.g., pollinator 100, etc.), the up and down buttons may be pressed to change the vibrational speed of the pollinators. If the pollinator only has a single speed (e.g., pollinator 200, etc.), then this step would not apply.

The off button may be pressed to stop the vibrating wand. The pollinator may be returned to and placed in the charging base on a flat surface when not in use. An electrical adapter or USB connector plugged into a computer may be used for providing electrical power for charging the pollinator. Preferably, the pollinator is charged sixteen hours prior to first use. The pollinator includes an LED light that flashes when the pollinator is charging and is continuous when the pollinator is on. Some exemplary embodiments of a handheld vibrating pollinator (e.g., pollinator 200, etc.) may be configured for use with non-rechargeable batteries (e.g., AA battery, etc.). In which case, the recharging steps would not be applicable and the battery(ies) would be replaced when necessary in these alternative embodiments.

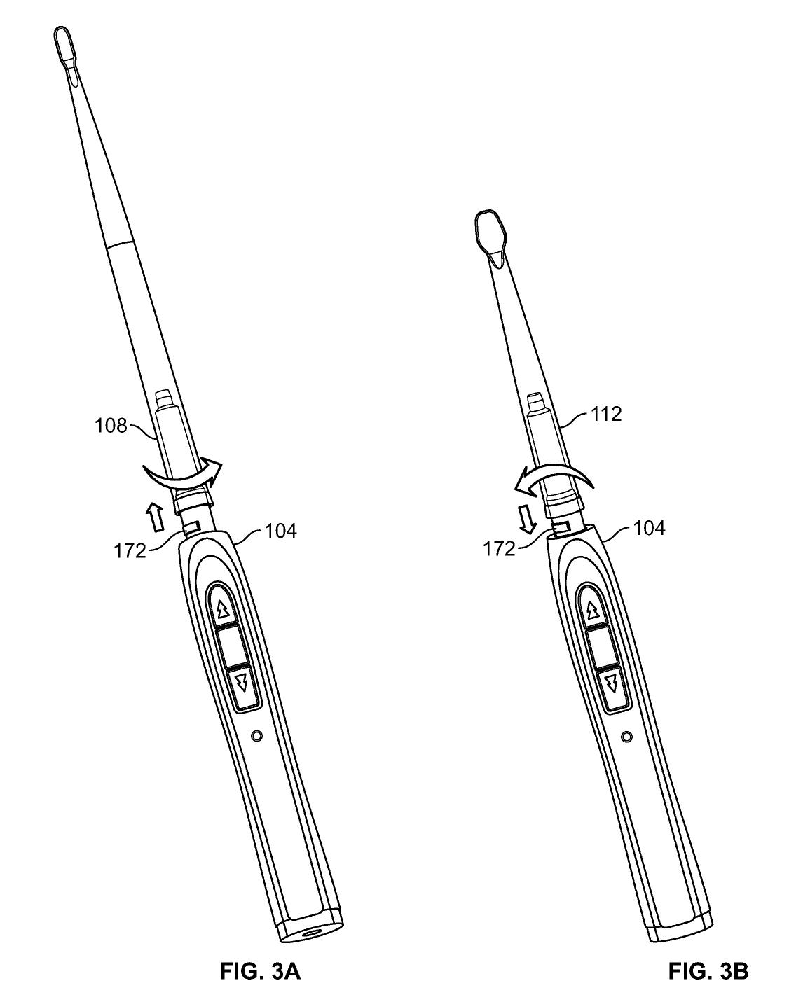

In exemplary embodiments that have more than one wand (e.g., pollinator 100, etc.), the wand may be changed as follows. First, the pollinator is turned off by pressing the off button if it is not already off. As shown in FIG. 3A, the wand 108 currently installed on the handle portion 104 of the pollinator 100 may be removed, e.g., by rotating the wand 108 one quarter turn to the right and lifting the wand 108 off the pollinator handle 104. The new wand 112 may then be installed by aligning a groove on the wand 112 with a ridge or protruding portion 172 of the handle portion 104, and then by gently rotating the wand 112 one-quarter turn left until the wand 112 clicks in place.

In exemplary embodiments that include a single removable wand, the wand may be removed and later reattached by the same process illustrated in FIGS. 3A and 3B. For example, and with reference to FIGS. 10 and 11, the wand 208 may be removed from the handle portion 204 of the pollinator 200 by rotating the wand 208 one quarter turn to the right and lifting the wand 208 off the pollinator handle 204. The wand 208 may be reinstalled by aligning a groove on the wand 208 with a ridge or protruding portion 272 (FIG. 11) of the handle portion 204, and then by gently rotating the wand 208 one-quarter turn left until the wand 208 clicks in place. Other exemplary embodiments may include a single wand that is fixedly attached to the handle portion. In such alternative embodiments, the steps about changing, removing, or reinstalling a wand would not be applicable.

In an exemplary embodiment, a pollinator may be configured to shut off after 2 minutes, and the pollinator may be configured to memorize the last speed setting used. The pollinator may be left on the charging base and plugged in when not in use.

Garden productivity may be improved by using the pollinator every other day between the hours of 11:00 am and 2:00 pm. Additional improvements may be realized by pollinating when the weather is dry and the air temperature is between 70 and 82 degrees Fahrenheit.

By way of example, a particular exemplary embodiment of a pollinator (e.g., pollinator 100 (FIG. 1), etc.) includes the following features: 5 speed electric pollinator with sonic speeds of 29,000-44,000 vibrations per minute, a vibration reducing handle, two vibrating wands having respective lengths of 11 cm and 18 cm, a pollen capture spoon, a Ni-MH rechargeable battery, AC and USB connectors, water resistant, UL certified, and RoHS compliant. Each one of the above features is not required or necessary for all embodiments, as other embodiments may include less than all of these features or none of them. For example, an alternative exemplary embodiment (e.g., pollinator 200 (FIG. 10), etc.) includes the following features: a single speed electric pollinator with sonic speeds of 38,000 vibrations per minute, a vibration reducing handle, a single vibrating wand having a length of 18 cm, a pollen capture spoon, battery powered (e.g., via one AA battery, etc.), water resistant, and FDA certified.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms (e.g., different materials, etc.), and that neither should be construed to limit the scope of the disclosure. In some example embodiments, well-known processes, well-known device structures, and well-known technologies are not described in detail. In addition, advantages and improvements that may be achieved with one or more exemplary embodiments of the present disclosure are provided for purpose of illustration only and do not limit the scope of the present disclosure, as exemplary embodiments disclosed herein may provide all or none of the above mentioned advantages and improvements and still fall within the scope of the present disclosure.

Specific dimensions, specific materials, and/or specific shapes disclosed herein are example in nature and do not limit the scope of the present disclosure. The disclosure herein of particular values and particular ranges of values (e.g., different vibrational frequency ranges, etc.) for given parameters are not exclusive of other values and ranges of values that may be useful in one or more of the examples disclosed herein. Moreover, it is envisioned that any two particular values for a specific parameter stated herein may define the endpoints of a range of values that may be suitable for the given parameter (i.e., the disclosure of a first value and a second value for a given parameter can be interpreted as disclosing that any value between the first and second values could also be employed for the given parameter). Similarly, it is envisioned that disclosure of two or more ranges of values for a parameter (whether such ranges are nested, overlapping or distinct) subsume all possible combination of ranges for the value that might be claimed using endpoints of the disclosed ranges.

The terminology used herein is for the purpose of describing particular example embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items. The term “about” when applied to values indicates that the calculation or the measurement allows some slight imprecision in the value (with some approach to exactness in the value; approximately or reasonably close to the value; nearly). If, for some reason, the imprecision provided by “about” is not otherwise understood in the art with this ordinary meaning, then “about” as used herein indicates at least variations that may arise from ordinary methods of measuring or using such parameters. For example, the terms “generally”, “about”, and “substantially” may be used herein to mean within manufacturing tolerances.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath”, “below”, “lower”, “above”, “upper” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The foregoing description of the embodiments has been provided for purposes of illustration and description. It is not intended to be exhaustive or to limit the disclosure. Individual elements, intended or stated uses, or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A pollinator comprising:
a spoon for capturing pollen released from a flower;
a handle portion operable for generating vibrations;
at least one wand configured to be installed on the handle portion, whereby the at least one wand is operable for transferring the vibrations generated by the handle portion to a flower for causing pollen to be released from the flower for capture by the spoon.

2. The pollinator of claim 1, wherein the at least one wand is configured to reduce an amplitude of the vibrations generated by the handle portion that travel along the at least one wand when installed on the handle portion.

3. The pollinator of claim 1, wherein the handle portion is operable for generating at least 30,000 vibrations per minute.

4. The pollinator of claim 1, wherein:
the handle portion is operable for generating vibrations within a range from 29,000 vibrations per minute to 44,000 vibrations per minute; or
the handle portion is operable for generating about 38,000 vibrations per minute.

5. The pollinator of claim 1, wherein the at least one wand comprises at least two wands having different configurations that are interchangeably installable on the handle portion, each said wand configured to reduce an amplitude of the vibrations generated by the handle portion that travel therealong when installed on the handle portion.

6. The pollinator of claim 1, wherein:
the pollinator further comprises a recharging base unit having an opening therein for receiving a lower portion of the handle portion;
the handle portion includes a battery compartment for one or more rechargeable batteries for electrically powering the pollinator; and
the recharging base unit is configured to recharge the one or more rechargeable batteries within the battery compartment when the lower portion of the handle portion is within the opening of the recharging base unit and the recharging base unit is connected to an external electrical power source.

7. The pollinator of claim 6, wherein the recharging base unit is electrically powered by a USB connector, which is pluggable into a USB port of a computer or electric wall AC adapter.

8. The pollinator of claim 7, wherein:
the handle portion comprises vibration dampening means;
the at least one wand comprises at least two wands having different configurations that are interchangeably installable on the handle portion, each said wand configured to reduce an amplitude of the vibrations generated by the handle portion that travel therealong when installed on the handle portion;
the recharging base unit further comprises openings for respectively receiving lower portions of the spoon and one of the two wands that is not installed on the handle portion; and
the handle portion is operable for selectively generating different speeds of vibrations ranging from 29,000 to 44,000 vibrations per minute.

9. The pollinator of claim 1, further comprising a base unit having openings therein for respectively receiving lower portions of the handle portion and the spoon for storing the pollinator when not in use.

10. The pollinator of claim 9, wherein:
the at least one wand comprises at least two wands having different configurations that are interchangeably installable on the handle portion; and
the base unit further comprises an opening therein for receiving a lower portion of one of the two wands that is not installed on the handle portion.

11. The pollinator of claim 9, wherein:
the handle portion comprises vibration dampening means;
the handle portion is operable for generating at least one speed of vibrations at about 38,000 vibrations per minute; and
the handle portion includes a battery compartment for one or more batteries for electrically powering the pollinator.

12. The pollinator of claim 1, wherein:
the handle portion includes a battery compartment for one or more rechargeable or non-rechargeable batteries for electrically powering the pollinator; and/or
the handle portion is operable for generating sonic vibrations that imitate high frequency vibrations made by a bee's wings during pollination; and/or
the at least one wand includes an end portion having a textured, roughened, and/or matt surface.

13. A pollination device comprising
a pollen capture device for capturing pollen released from a flower;
a handle portion operable for generating sonic vibrations;
at least one member configured to be installed on the handle portion, whereby the at least one member is operable for transferring the sonic vibrations generated by the handle portion to a flower for causing the flower to release pollen for capture by the pollen capture device.

14. The pollination device of claim 13, wherein the at least one member is configured to reduce an amplitude of the sonic vibrations generated by the handle portion that travel along the at least one member when installed on the handle portion, so that the sonic vibrations do not damage the flower.

15. The pollination device of claim 13, wherein:
the handle portion is operable for generating at least 30,000 vibrations per minute; and/or
the handle portion is operable for generating vibrations within a range from 29,000 vibrations per minute to 44,000 vibrations per minute; and/or
the handle portion is operable for generating about 38,000 vibrations per minute.

16. The pollination device of claim 13, wherein the at least one member comprises at least two wands having different configurations that are interchangeably installable on the handle portion, each said wand configured to reduce an amplitude of the vibrations generated by the handle portion that travel therealong when installed on the handle portion.

17. The pollination device of claim 13, wherein:
the pollination device further comprises a recharging base unit having an opening therein for receiving a lower portion of the handle portion;
the handle portion includes a battery compartment for one or more rechargeable batteries for electrically powering the pollination device; and
the recharging base unit is configured to recharge the one or more rechargeable batteries within the battery compartment when the lower portion of the handle portion is within the opening of the recharging base unit and the recharging base unit is connected to an external electrical power source.

18. The pollination device of claim 13, further comprising a base unit having openings therein for respectively receiving lower portions of the handle portion and the spoon for storing the pollination device when not in use.

19. The pollination device of claim 13, wherein:
the handle portion includes a battery compartment for one or more rechargeable or non-rechargeable batteries for electrically powering the pollination device; and/or
the handle portion is operable for generating sonic vibrations that imitate high frequency vibrations made by a bee's wings during pollination; and/or
the at least one member includes an end portion having a textured, roughened, and/or matt surface.

20. A pollinator comprising:
a spoon for capturing pollen released from a flower;
a handle portion operable for generating sonic vibrations that are within a range from 29,000 vibrations per minute to 44,000 vibrations per minute; and
at least one wand operable for transferring the sonic vibrations generated by the handle portion to a flower for causing the flower to release pollen, the at least one wand configured to reduce an amplitude of the sonic vibrations that travel therealong so that the sonic vibrations do not damage the flower.
